(19) United States
(12) Patent Application Publication
(10) Pub. No.: US 2026/0295289 A1
(43) Pub. Date: Oct. 1, 2026

(54) PHOTOTHERAPY DEVICE FOR LIGHT EMISSION

(71) Applicant: Aesthetic Technology Ltd, Warrington (GB)

(72) Inventors: Dale Needham, Warrington (GB); Ben Rawlings, Warrington (GB); Huw Anthony, Warrington (GB); Adam Thomas, Warrington (GB)

(21) Appl. No.: 18/996,337

(22) PCT Filed: Aug. 18, 2023
(86) PCT No.: PCT/GB2023/052173

§ 371 Date: Jan. 17, 2025

Related U.S. Application Data
(60) Provisional application No. 63/371,959, filed on Aug. 19, 2022.

(51) Int. Cl. A61N 5/06

(52) U.S. Cl. CPC A61N 5/0616; A61N 2005/0652; A61N 2005/0664

(57) ABSTRACT

A phototherapy device to deliver light emission to a person. The device comprises an array of LEDs mounted at an LED printed circuit board. At least one fan is mounted internally within the device to drive a cooling air flow stream through the internal electronic components to effectively cool the various electronic components.

### FIELD OF INVENTION

The present concept relates to a phototherapy device to deliver light emission to a person, and in particular, although not exclusively, to a phototherapy device and method in which internal electronic components of the device are cooled effectively by at least one air flow stream driven through the device.

### BACKGROUND

Phototherapy, being ‘treatment with light’ is the application of light energy to stimulate or regulate biological processes with proven therapeutic effects. The effective mechanism is a natural response similar to that of plant photosynthesis through a process known as photobiomodulation. LED Phototherapy (i.e., phototherapy using light-emitting diodes (LEDs) to deliver the light) is well evidenced for its regenerative and anti-inflammatory benefits without creating trauma making it safe and suitable for all skin types. The treatment involves exposing the skin to low levels of beneficial light energy from the visible and infrared part of the light spectrum. Specific wavelengths interact with biological systems and activate key cell receptors which consequently trigger a transfer of light energy to cellular energy. Skin cells that are energised function better and can renew faster to promote youthful, healthy, and radiant skin. With a shift towards non-invasive treatments, LED Phototherapy offers a progressive alternative to more aggressive procedures which carry a higher risk of adverse response, discomfort, and downtime. LED energy delivered via spectrally pure wavelengths stimulate the skin's own repair mechanism, correct problem skin conditions and help to restore optimum skin function.

LED Phototherapy is now recognised as an essential modality for skin care practitioners and is well evidenced for the safe and effective treatment of a wide range of inflammatory and problematic skin conditions, accelerated wound healing and ageing skin indications.

WO 2006/020602 A1 describes a skin treatment phototherapy device formed as a pen, a face mask or a desk lamp. The device includes an array of LEDs in particular multi-coloured LEDs for emitting multiple wavelengths of light for skin treatment.

WO 2014/131115 A1 describes a phototherapy device having first and second light sources to create different respective emission spectra in the blue and ultraviolet regions of the electromagnetic spectrum.

However, there is a continued need for improved LED-based phototherapy devices with improved optical and energy efficiency.

### SUMMARY OF THE INVENTION

It is an objective of the present concept to provide a phototherapy device for the treatment of various dermatological conditions having enhanced operational efficiency and extended component lifetime.

It is a further specific objective to provide a LED based phototherapy device having enhanced resistance to lumen degradation over time. It is a further specific objective to provide a phototherapy device with a reduced running temperature, more consistent light output and improved treatment results. It is a further objective to provide a portable and lightweight device.

The objectives are achieved via a phototherapy device configured specifically with real-time active cooling of the various electronic components. According to the various implementations, the cooling effect is achieved using at least one fan configured to drive a cooling air flow stream through the device and onto the various electronic components to be cooled. In particular, the present device utilises a heatsink thermally coupled to an array of light emitting LEDs mounted at an LED printed circuit board (PCB). At least one fan or fan assembly is configured to direct an air flow stream onto and into the heatsink to enhance heat dissipation from the LEDs and in particular the LED PCB. Additionally, the present cooling system allows for a mechanically lightweight device offering portability and ease of use.

In particular, the inventors provide means and method for the effective thermal management of LEDs within the device by forced air cooling and active component temperature management. The present system may comprise temperature sensors, software and/or firmware operative for the real-time monitoring of the temperature of the various electronic components so as to provide a responsive system adapted to change a magnitude of cooling of the electronic components in real-time. Accordingly, the present device comprises enhanced thermal management that in turn provides improved optical power performance and extended device lifetime.

The present system via hardware and/or software is configured to selectively adjust i.e. increase and/or decrease, a volume and/or speed of air flowing internally through the device in direct response to the identified temperature of one of more components and in particular electronic components of the device. In particular, the present concept is configured to monitor the temperature of the LEDs and to increase and/or decrease the rotational speed of the at least one fan to increase and/or decrease the air flow rate and/or volume flowing through the device that is effective to cool the LEDs. Such a configuration is effective to maintain the desired and optimal power output of the LEDs so as to achieve a desired uniform and consistent light radiation from the device for the effective and controlled treatment of a patient by phototherapy. Such an arrangement, in turn, provides improved treatment and in particular less heat created during treatment. This enables light to penetrate the skin with improved management of the optical power output during photodynamic therapy (PDT) treatments.

In specific embodiments, the present system may be adapted to control and regulate the output of the LEDs on-demand and in response to the temperature of one or more components of the apparatus. For example, where an electronic component has reached a threshold temperature, the present system may be configured to react by reducing the power output at one or more selected LEDs and/or increasing a rotational speed of an air flow driving fan to enhance heat dissipation from the hot component. Additionally, the present system also improves patient safety by appropriate temperature management of the LEDs and other operational components providing a treatment device that is less likely to cause burns or even present a fire hazard.

According to a first aspect of the present concept there is provided a phototherapy device to deliver light emission to a person comprising: an LED assembly comprising a plurality of LEDs mounted at a first face of an LED printed circuit board; a heatsink mounted against a second face of the LED printed circuit board; a housing to at least partially contain the LED printed circuit board and the heatsink, the housing having first and second spatially separated air flow vents to allow a flow of air into and out of the housing; at least one fan positioned in air flow direction between the vents to drive a flow of air through the device; wherein at least a portion of the heatsink is positioned in the air flow direction between the vents so as to be in a path of the flow of air through the device.

The housing of the various electronic components may be provided with a funnel-type configuration that, in combination with the at least one fan creates air flow turbulence/streams within the device for the efficient heat transfer and dissipation at the heatsink. In particular, the present housing having the respective vents is configured to create a relatively tight air flow channel around the heatsink and other electronic components to enhance the function of the at least one fan to dissipate the heat energy.

The present system and in particular the heatsink component provides a balance between surface area and airflow through the device. Importantly, the heatsink includes a surface area size that is sufficient to draw heat whilst providing sufficient spacing/volume between the heat dissipation fins. This allows cooling air to dissipate the heat generated from the LEDs and/other electronic operational components.

The present device via the cooling configuration is effective to reduce the temperature of the LEDs and an ambient temperature at the housing and/or regions of the housing (both external and internal). Due to the increased air flow, the present device is provided with lower general running temperatures to improve the optical performance and optical stability of light generated by the LEDs. Additionally, the present device provides consistent power per second over the course of treatment due to the enhanced thermal management of the LEDs. The greater LED running efficiency also minimises lumen degradation over time to enhance the operational lifetime of the device and consistency of treatment over time.

The device optionally comprises an array of identical LEDs. Optionally, according to various embodiments, the LEDs may comprise different types configured to emit light of different wavelengths and/or a different intensity.

Preferably, the heatsink comprises a base and a plurality of fins projecting outwardly from the base, the fins comprising respective panes positioned side-by-side over the base to define air flow channels between the panes. Optionally, a width of each of the channels is greater than a thickness of each of the panes. Optionally, the thickness of each pane is in a range 5 to 30%, 5 to 25% or 10 to 20% of the width of each channel.

Optionally, the at least one fan is positioned adjacent an entry end or exit end of the channels. Preferably, the channels are elongate between the entry and exit ends and wherein a rotational axis of the at least one fan is aligned generally parallel with the length of the elongate channels. Preferably, the at least one fan is positioned in an air flow direction between one of the vents and the entry or exit ends of the channel. Additionally, the at least one fan is aligned with the entry or exit turbulence during airflow transfer.

Preferably, the device comprises a plurality of fans positioned side-by-side and laterally across a width of the heatsink. Optionally, the device may comprise two, three, four or five fans for example. Optionally, the device comprises at least one fan at a first lengthwise end of the heatsink and/or at least one fan at a second lengthwise end of the heatsink. Optionally, according to further embodiments, the device comprises at least a first fan at or adjacent a region of a first lengthwise end of the heatsink and at least one fan at or adjacent to a region at a second lengthwise end of the heatsink. Accordingly, in such an embodiment, the fans are configured to drive an air flow stream into the device (via at least one fan) and to drive an air flow stream out of the device (via at least one additional fan).

Preferably, the device comprises a thermal conductivity pad positioned between the second face of the LED printed circuit board and the heatsink. The pad may comprise any suitable material of enhanced thermal conductivity. Optionally, the pad may comprise paraffin wax, a silicone-based material or any polymeric material. Optionally, the material of the pad comprises a metal or metal-based material. Optionally, a thermal conductivity pad comprises a thermal conductivity in a range 5 to 15 W/m·k. The thermal conductivity, as described herein may be measured by steady-state or transient measurement techniques where steady-state involves thermal conductivity measurements at a state of a material once a steady-state temperature profile has been reached whilst transient measurement operates according to an instantaneous state of a system during the approach to steady-state as will be appreciated by those skilled in the art. Optionally, the thermal conductivity is measured in accordance with ISO 22007-2. Optionally, a thickness of the pad is in a range 0.1 to 0.25 mm or 0.1 to 0.2 mm. Optionally, the heatsink comprises a metal or metal-based material. Optionally, the heatsink comprises aluminium or copper.

Optionally, the device further comprises a LED driver printed circuit board. Optionally, the LED driver board comprises an array of electronic components to control the operative state of the various electronic components of the device including in particular the LEDs and the fans. The LED driver board may comprise firmware and software including control utilities and modules for the control of the various electronic components. In particular, the driver board may comprise a processor, a data storage utility, a comms (wired and wireless) interface, a user interface, a display, a power source (battery), a power port, an external port to connect the device to a network, other components/devices and/or mains power.

Optionally, the LED drive printed circuit board may be positioned in an air flow direction between the vents and opposite the channels at a region between the entry and exit ends of the channels.

Optionally, the device further comprises an air flow shroud mounted at a position between the LED driver printed circuit board and the heatsink to at least partially guide air flow onto the LED driver printed circuit board.

Optionally, the device may comprise at least one secondary fan positioned at a region of the first face of the LED printed circuit board and the housing, the housing comprising at least one secondary vent to allow a secondary flow of air driven by the at least one secondary fan to exit the housing proximate to the plurality of LEDs. Preferably, the device comprises at least a pair of secondary fans positioned proximal to a face of the device intended to be forward facing and opposite a person to be treated.

Optionally, the at least one secondary fan is physically separated from the at least one fan positioned in air flow direction between the vents to provide that the at least one secondary flow of air is partitioned and/or separated from the air flow flowing through the device between the vents. Advantageously, the primary airflow (to cool the LEDs) and the secondary airflow (for patient cooling) are airflows are physically/mechanically separated. This provides that secondary airflow is unaffected by the primary flow and is always cool as supplied/directed to a patient.

Optionally, the device comprises at least one temperature sensor positioned at any one or a combination of: the LEDs; the LED printed circuit board; the LED driver printed circuit board; a region inside the housing; the heatsink; at least one of the panes. Preferably, the sensors are connected electronically to the control components of the device.

According to a further aspect of the present concept there is provided a method of delivering light emission to a person comprising: directing light emitted from an LED assembly to a person; dissipating heat generated by the LED assembly using a heatsink mounted against the LED printed circuit board; cooling the heatsink and/or the LED assembly by driving a flow of air over and/or through a heatsink during the light emission from the LEDs using at least one fan.

### BRIEF DESCRIPTION OF DRAWINGS

A specific implementation of the present invention will now be described, by way of example only, and with reference to the accompanying drawings in which:

FIG. 1 is a perspective view of a phototherapy device comprising an array of LEDs mounted at a housing according to a specific implementation of the present concept;

FIG. 2 is a plan view of an upper side of the phototherapy device of FIG. 1;

FIG. 3 is a first end elevation view of the device of FIG. 2;

FIG. 4 is second end elevation view of the device of FIG. 2;

FIG. 5 is an expanded perspective view of the various internal components of the phototherapy device of FIGS. 1 to 4;

FIG. 6 is an underside view of the phototherapy device of FIG. 5;

FIG. 7 is a perspective cross sectional view through A-A of the device of FIG. 6;

FIG. 8 is a perspective cross sectional view through B-B of FIG. 9;

FIG. 9 is an underside view of the phototherapy device of FIG. 6;

FIG. 10 is a further underside view of the phototherapy device of FIG. 6;

FIG. 11 is a cross sectional view through C-C of FIG. 10;

FIG. 12 is an end perspective view of a heatsink component forming part of the assembly of the phototherapy device of FIG. 5.

### DETAILED DESCRIPTION OF PREFERRED EMBODIMENT OF THE INVENTION

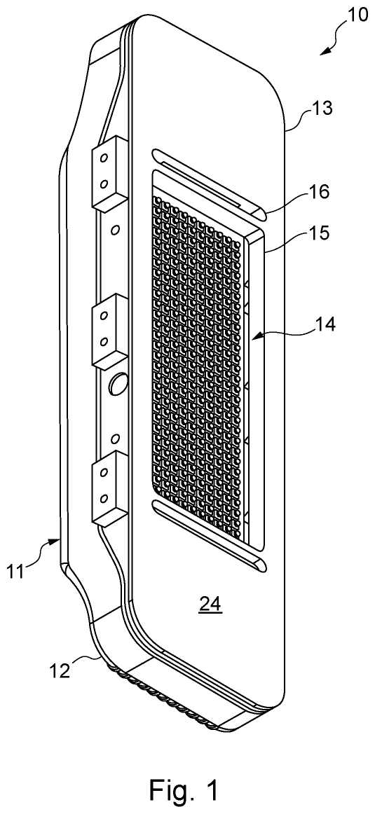

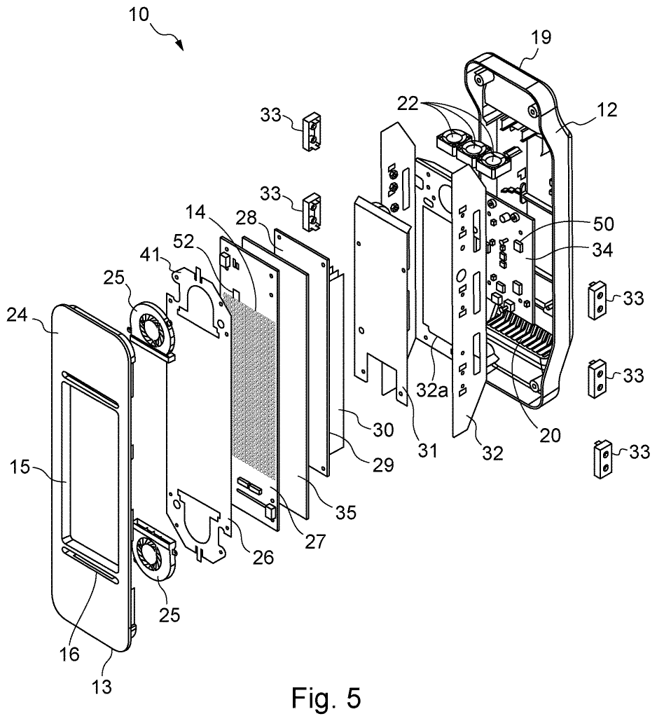

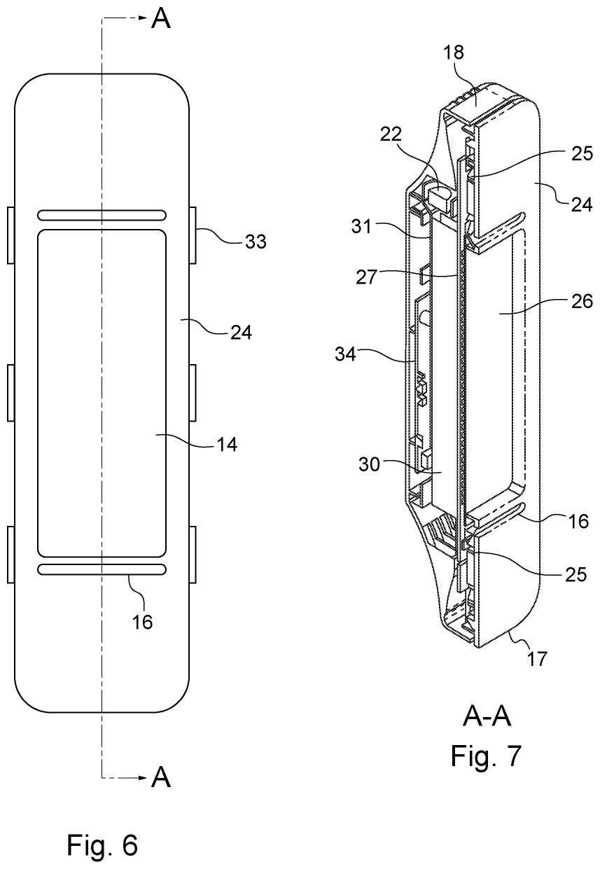

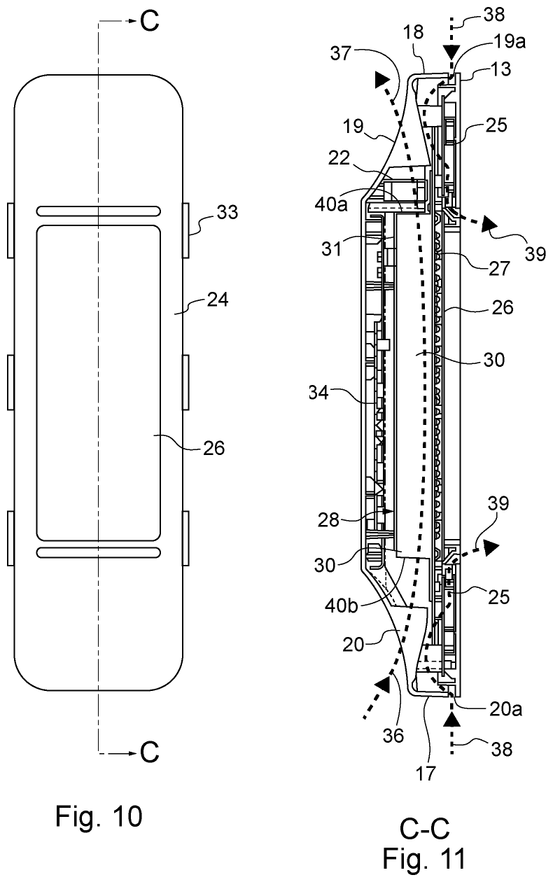

Referring to FIG. 1, a phototherapy device 10 comprises a housing indicated generally by reference 11. Housing 11 comprises a first rear casing part 12 and a front plate part 13. Parts 12 and 13 are releasably coupled together by suitable mountings and/or clips. Part 13 comprises a generally planar main face 24 and a window 15 having a generally rectangular shape profile formed within face 24. An array of LEDs 14 are mounted internally within device 10 and are at least partially exposed through window 15. A pair of apertures 16 are provided at part 13 adjacent each lengthwise end of window 15. According to the specific implementation, device 10 comprises a plurality of fans configured to create and maintain a plurality of independent air flow streams internally through device 10. In particular, at least one air flow stream is configured to cool internal electronic components associated with the light emission generated from LEDs 14 whilst at least one secondary air flow is capable of flowing out of device 10 via secondary apertures 16 to provide a cooling effect to a person that is being treated by the light emitted by LEDs 14. The various internal electronic components associated with the function of LEDs 14 are described referring to FIGS. 5, 7, 8 and 10.

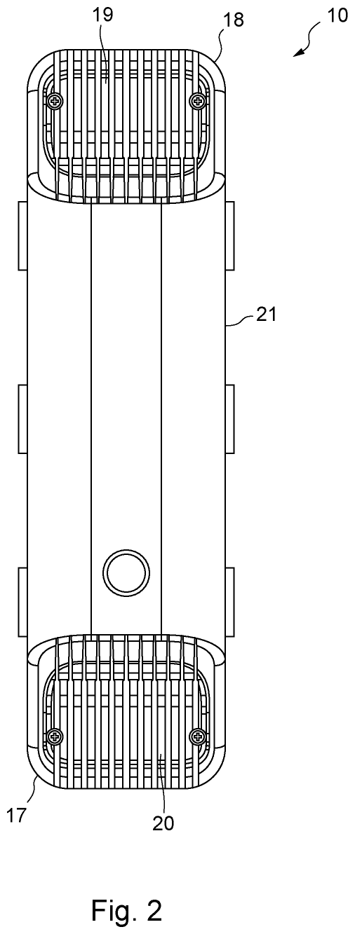

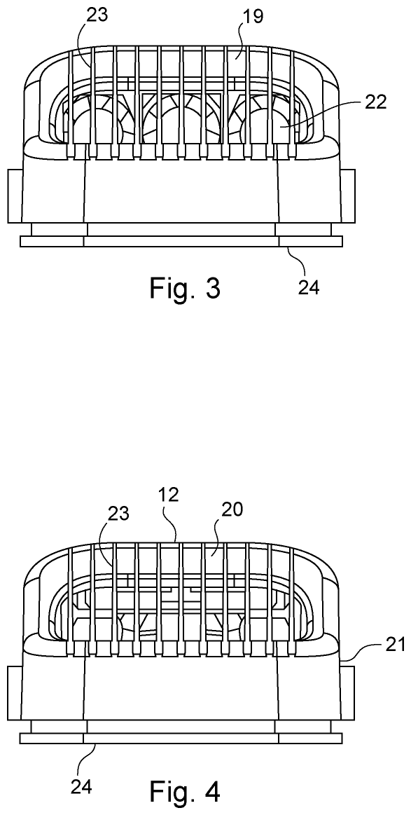

Referring to FIGS. 2 to 4, housing 11 comprises first and second air flow vents 19, 20. Each vent 19, 20 is positioned at or towards each lengthwise end 18, 17 of device 10. Device 10 is generally elongate between ends 17, 18 and comprises lateral sides 21 extending lengthwise between ends 17, 18. Each vent 19, 20 comprises a respective grill 23 formed from ribs or flanges that extend across each of the vents 19, 20. Grills 23 function to prevent items or objects passing into the internal regions of device 10 undesirably via vents 19, 20.

Referring to FIG. 5, housing 11, having respective rear and front parts 12, 13, comprises an internal chamber to house the various physical and electronic components associated with the operation and function of light emission from device 10 that in use is adapted to deliver light radiation from LEDs 14 to a person being treated. Device 10 comprises within its internal chamber, an LED printed circuit board (PCB) 27 on which are mounted the plurality of LEDs 14. A clear LED screen 26 is positioned adjacent a front face of LED PCB 27. A pair of secondary fans 25 are mounted within respective slots 41 formed within the end regions of screen 26. Fans 25 comprise a relatively low profile configuration so as to be aligned with the general plane of screen 26. LED PCB 27, screen 26 and fans 25 are positioned against an internal face of housing part 13 so as to present the array of LEDs 14 at window 15 through screen 26.

A heatsink indicated generally by reference 28 is positioned at an opposite second face of LED PCB 27. A thermal conductive pad 35 is sandwiched intermediate heatsink 28 and LED PCB 27. Pad 35 comprises a suitable high thermal conductivity material. Optionally, the thermal conductivity pad comprises a thermal conductivity in a range 8 to 12 W/m·k. Optionally, a thickness of pad 35 is around 0.1 to 0.2 mm. Heatsink 28 comprises a base plate 29 being generally rectangular and having a surface area approximately equal to a corresponding surface area of the second face of LED PCB 27 against which heatsink 28 is positioned. A plurality of fins 30 project laterally outward from base plate 29 to form an array of heatsink fins being aligned generally perpendicular to base plate 29 and LED PCB 27. An air funnel shroud 31 comprises a generally planar body and is positioned adjacent the lengthwise extending edges of fins 30 such that fins 30 project laterally between shroud 31 and heatsink base plate 29. A pod chassis 32 is positioned adjacent shroud 31 at an opposite side relative to heatsink 28. Chassis 32 comprises an aperture 32a. An LED driver printed circuit board (PCB) 34 is mountable against an internal facing side of rear casing part 12. Board 34 is exposed through aperture 32a so as to be positioned opposite one face of shroud 31. A plurality of air flow fans 22 are mountable against rear casing part 12 at a position proximal to vent 19. In particular, with the various components of FIG. 5 assembled and contained within housing 11, fans 22 are positioned in an air flow direction intermediate vent 19 and a lengthwise end of heatsink 28 and in particular the lengthwise ends of fins 30. The various components are retained securely within housing 11 by mounting blocks 33 that inter-engage with lateral sides of chassis 32 so as to releasably lock together housing parts 12, 13.

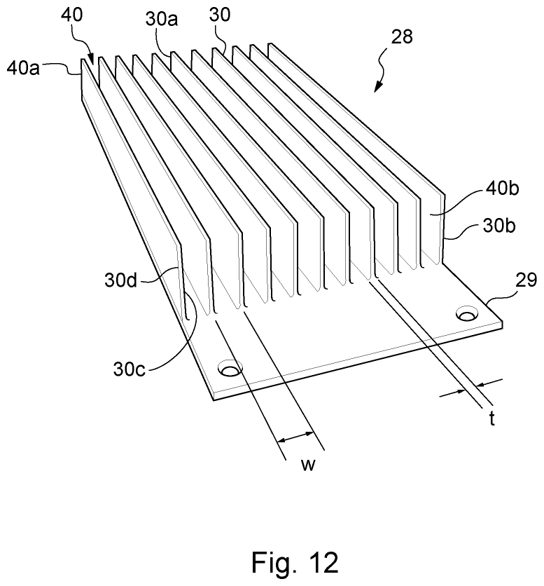

Referring to FIG. 12, the plurality of heatsink fins 30 are arranged side-by-side at base plate 29. Each of the fins 30 are elongate in a lengthwise direction of device 10 between respective ends 17, 18. In particular, each fin 30 comprises a first lengthwise end 30a and a second lengthwise end 30b. Each fin 30 is formed as an elongate plate-like body having respective side faces 30c, 30d that define primarily, elongate channels 40. Accordingly, channels 40 comprise first lengthwise ends 40a and second lengthwise ends 40b. With heatsink 28 mounted within housing 11 and against LED PCB 27, fans 22 are positioned between vent 19 and the respective ends 30a, 40a of the heatsink fins 30 and channels 40. Accordingly, fans 22 are positioned in an air flow direction between vent 19 and the channel ends 40a.

According to the specific implementation, a width w of each channel 40 is greater than a corresponding thickness t of each plate-like fin 30. In particular, and according to the specific implementation, t is in a range 10 to 20% of w. Accordingly, the air flow passageways through heatsink 28 are maximised due to the enhanced volume available for air flow travel between fins 30.

In use, with LEDs 14 powered by a suitable power source, such as a battery (not shown), driver PCB 34 is effective to control the function and operation of LEDs 14 at LED PCB 27. Light of an appropriate/desired wavelength is emitted from LEDs 14 through window 15 via screen 26. The operation of the secondary cooling fans 25 is achieved via driver PCB 34 and/or independent control components (not shown) housed within housing 11.

Referring to FIGS. 5, 8 and 11, fans 22 are orientated such that their respective rotation axes 53 are aligned parallel with elongate air flow channels 40. With fans 22 mounted in this orientation and immediately adjacent channel ends 40a, when energised, fans 22 create and maintain an air flow stream through device 10. In particular, and referring to FIG. 11, fans 22 are effective to draw-in a stream of air 36 into device 10 via vent 20. The air flow streams then pass-through channels 40 and are effective to cool heatsink 28 and in turn LED PCB 27 and LEDs 14. An exit air flow stream 37 is then propelled from the internal region of device 10 via vent 19 positioned proximal to fans 22. Additionally, due to the respective mounting position of driver PCB 34, chassis 32 and shroud 31, the cooling air flow stream 36 also functions to cool all electronic components mounted at driver PCB 34 in addition to the LEDs 14 and other electronic components mounted at and associated with LED PCB 27.

Fans 25 are operative to drive streams of air 38 into the housing 11 via small secondary vents 19a, 20a poisoned at each housing end 17, 18. Respective air flow streams 39 are driven (by fans 25) and directed out of the device 10 via apertures 16 to provide a cooling stream of air to a person that is being treated.

The heat dissipation and cooling effect provided by heatsink fins 30, fan 22 and vents 19, 20 relative to the LED PCB 27 and LEDs 14, is enhanced by thermal conductive pad 35.

Pad 35 is effective for the efficient thermal conductivity between LED PCB 27 and heatsink 28.

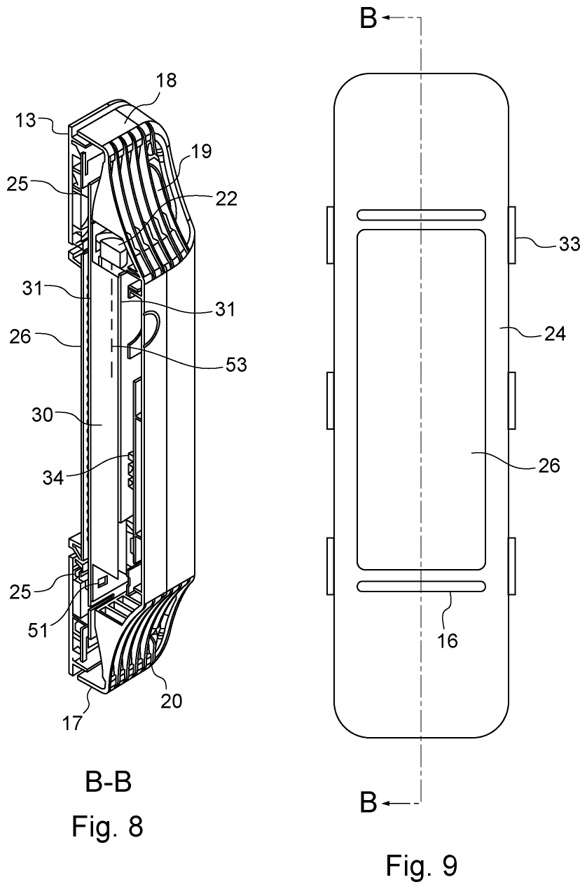

Device 10 comprises at least one temperature sensor. In particular, and according to a specific implementation, a temperature sensor 50 (FIG. 5) is provided at driver PCB 34, a temperature sensor 51 (FIG. 8) is provided at heatsink 28 and a temperature sensor 52 (FIG. 5) is provided at the array of LEDs 14 and LED PCB 27. Temperature sensors 50, 51, 52 are provided in electronic communication with driver PCB 34 and/or other suitable control components so as to provide real-time temperature sensing during light emission from LEDs 14. Device 10, optionally at driver PCB 34 comprises suitable control software for the thermal management of the various electronic components of device 10 in real-time and responsive to data output and collected from temperature sensors 50, 51, 52. Accordingly, device 10 via software and/or the other electronic components as described and detailed herein, is configured to control in real-time the magnitude of cooling applied to the LEDs 14 dependent upon the observed temperature of selected internal electronic components such as the temperature at driver PCB 34, heatsink 28, LEDs 14. In particular decrease, suitable control software, operative in communication with fans 22, is configured to control a rotational speed of fans 22 so as to selectively adjust in real-time an angular velocity of fans 22 and in-turn the air flow velocity and/or volume of air passing through device 10 (streams 36, 37), in response to the temperature of any one or a combination of the electronic components described and detailed herein.

1. A phototherapy device to deliver light emission to a person comprising:
an LED assembly comprising a plurality of LEDs mounted at a first face of an LED printed circuit board;
a heatsink mounted against a second face of the LED printed circuit board;
a housing to at least partially contain the LED printed circuit board and the heatsink, the housing having first and second spatially separated air flow vents to allow a flow of air into and out of the housing;
at least one fan positioned in air flow direction between the vents to drive a flow of air through the device;
wherein at least a portion of the heatsink is positioned in the air flow direction between the vents so as to be in a path of the flow of air through the device.

2. The device as claimed in claim 1 wherein the heatsink comprises a base and a plurality of fins projecting outwardly from the base, the fins comprising respective panes positioned side-by-side over the base to define air flow channels between the panes.

3. The device as claimed in claim 2 wherein a width of each of the channels is greater than a thickness of each of the panes.

4. The device as claimed in claim 3 wherein the thickness of each pane is in a range 5 to 30%, 5 to 25% or 10 to 20% of the width of each channel.

5. The device as claimed in claim 2 wherein the at least one fan is positioned adjacent an entry end or exit end of the channels.

6. The device as claimed in claim 5 wherein the channels are elongate between the entry and exit ends and wherein a rotational axis of the at least one fan is aligned generally parallel with the length of the elongate channels.

7. The device as claimed in claim 6 wherein the at least one fan is positioned in an air flow direction between one of the vents and the entry or exit ends of the channel.

8. The device as claimed in claim 1 comprising a plurality of fans positioned side-by-side and laterally across a width of the heatsink.

9. The device as claimed in claim 1 further comprising a thermal conductivity pad positioned between the second face of the LED printed circuit board and the heatsink.

10. The device as claimed in claim 9 wherein the thermal conductivity pad comprises a thermal conductivity in a range 5 to 15 W/m·k.

11. The device as claimed in claim 9 wherein a thickness of the pad is in a range 0.1 to 0.25mm or 0.1 to 0.2 mm.

12. The device as claimed in claim 1 further comprising a LED driver printed circuit board.

13. The device as claimed in claim 5 wherein the LED driver printed circuit board is positioned in an air flow direction between the vents and opposite the channels at a region between the entry and exit ends of the channels.

14. The device as claimed in claim 12 comprising an air flow shroud mounted at a position between the LED driver printed circuit board and the heatsink to at least partially guide air flow onto the LED driver printed circuit board.

15. The device as claimed in claim 1 comprising at least one secondary fan positioned at a region of the first face of the LED printed circuit board and the housing, the housing comprising at least one secondary vent to allow a secondary flow of air driven by the at least one secondary fan to exit the housing proximate to the plurality of LEDs.

16. The device as claimed in claim 15 wherein the at least one secondary fan is physically separated from the at least one fan positioned in air flow direction between the vents to provide that the at least one secondary flow of air is partitioned and/or separated from the air flow flowing through the device between the vents.

17. The device as claimed in claim 2 comprising at least one temperature sensor positioned at any one or a combination of:
the LEDS;
the LED printed circuit board;
the LED driver printed circuit board;
a region inside the housing;
the heatsink;
at least one of the panes.

18. A method of delivering light emission to a person comprising:
directing light emitted from an LED assembly to a person;
dissipating heat generated by the LED assembly using a heatsink mounted against the LED printed circuit board;
cooling the heatsink and/or the LED assembly by driving a flow of air over and/or through a heatsink during the light emission from the LEDs using at least one fan.
